(19) United States
(12) Patent Application Publication
(10) Pub. No.: US 2016/0341723 A1
(43) Pub. Date: Nov. 24, 2016

(54) AU NANOPARTICLES ENCAPSULATED IN NANOCOMPOITES AND APPLICATIONS THEREOF IN RAPID DETECTION OF AN ANALYTE

(71) Applicants: Nahid Sarlak, Tehran (IR); Zeinab Sameri, Tehran (IR)

(72) Inventors: Nahid Sarlak, Tehran (IR); Zeinab Sameri, Tehran (IR)

(21) Appl. No.: 15/225,798

(22) Filed: Aug. 1, 2016

Related U.S. Application Data
(60) Provisional application No. 62/246,063, filed on Oct. 25, 2015.

(51) Int. Cl. G01N 33/543; G01N 21/78; G01N 33/558; G01N 33/553; G01N 33/532

(52) U.S. Cl. CPC G01N 33/54346; G01N 33/553; G01N 33/532; G01N 33/558; G01N 21/78; G01N 2021/7763

(57) ABSTRACT

A method of preparation for a detection pad for detecting an analyte. The method includes preparing a labeling substance including a plurality of metal nanoparticles encapsulated in a nanocomposite complex. The labeling substance may be conjugated with at least one analyte-binding partner to prepare a labeled detection reagent. The detection reagent may then be applied to a pad to prepare a reagent absorption pad. A control pad may then be prepared, and finally the control pad and conjugation pad may be bound together to form the detection pad.

### CROSS REFERRENCE TO RELATED DISCLOSURE

This disclosure claims the benefit of priority from pending U.S. Provisional Patent Application No. 62/246,063, filed on Oct. 5, 2015, and entitled “AU NANOPARTICLES ENCAPSULATED IN POLYMERIZED CARBON NANOTUBES AND APPLICATIONS THEREOF IN RAPID DETECTION OF VIRUSES” which is incorporated herein by reference in its entirety.

### TECHNICAL FIELD

The present invention generally relates to an assay for determining the presence of an analyte, and more particularly to a new approach toward the development of higher sensitive immunoassay-based dipsticks which make use of bio-functionalized nano-gold as a detection reagent, and even more particularly to utilizing CNT-g-PCA-Au for labeling antibodies in order to improve the sensitivity of the immunoassay-based strip test.

### BACKGROUND

The emerging research field of nanotechnology, the process to generate and manipulate nanomaterials, provides exciting new possibilities for advanced development of new labels for bioanalytical applications.

Methods of immune-chromatography analysis (hereinafter “IChA”) that use nanoparticles of colloidal gold as a label, make it possible to visually detect the compound under determination. This approach is called lateral flow immune-analysis (LDFs). A one-step test may be performed within a few minutes without the need for instrumentation and additional chemicals. Furthermore, results may be interpreted by non-specialists.

However, there is still a need in the art for higher signal intensity and better quantitative discrimination of the color-formation reaction based on label accumulation in LDFs. Since LFDs are designed for visual inspections, the type of label may be considered an important factor for successful development of user-friendly tests with sufficient sensitivity. Moreover, the high cost of the test strips is considered another drawback of this method. Therefore, there is a need in the art for development of a method for manufacturing higher quality labeling substances with higher sensitivity and lower costs.

### SUMMARY

The following brief summary is not intended to include all features and aspects of the present disclosure, nor does it imply that exemplary embodiment must include all features and aspects discussed in this summary.

Disclosed exemplary detection pad and method for its preparation for detecting the presence of an analyte in a strip test, for example immunoassay test strip, may include the following steps: preparing a labeling substance which contains a metal nanoparticle encapsulated nanocomposite complex; labeling of at least one analyte-binding partner with the labeling substance to prepare a labeled detection reagent; applying the prepared labeled detection reagent into a capture membrane to prepare a conjugation pad; preparing a control pad; and joining the control pad and the conjugation pad to obtain the detection pad.

In an exemplary implementation, a conjugation pad may be a main part of the detection pad, and may generally contain a detection reagent and the detection reagent may include a labeling substance which may be conjugated with the analyte-binding partners. The analyte-binding partners may be capable of specific binding with the analyte of interest and may be selected from the group consisting of: antigens and antibodies, fragments of antibodies, lectins, carbohydrates, hormones, hormone receptors, enzymes, enzyme substrates, vitamins, vitamin binding proteins, drugs, and receptors.

A labeling substance may include a complex in which a plurality of nanoparticles may be encapsulated into a nanocomposite. According to one exemplary implementation the nanocomposite may be carbon nanotubes which may be grafted with poly (citric acid). In some implementation, the metal nano particles may be gold nano particles (AUNPs). In some other implementation, a combination of gold nanoparticles with other metal nanoparticles, for example, silver nanoparticles may be used.

In another exemplary implementation, synthesizing the nanocomposite complex may comprise the steps of: synthesizing a nanocomposite which contain carbon nanotubes grafted with poly (citric acid); mixing a metal source with the synthesized nanocomposite containing carbon nanotube grafted with poly (citric acid) to provide a mixture; ultra-sonicating and stirring the mixture. The metal source may be a gold source, however, in some instance another nanoparticle may be used in some instance, a combination of gold nanoparticles and other metal source may be used.

In another exemplary implementation, synthesizing carbon nanotubes grafted with poly (citric acid) may comprises the step of: oxidizing carbon nanotubes; mixing the oxidized carbon nanotubes and monohydrated citric acid to synthesize carbon nanotubes grafted with poly (citric acid). Oxidizing the carbon nanotubes may be implemented by mixing the carbon nanotubes with nitric acid and sulfuric acid. The ratio of the nitric acid to the sulfuric acid is in the range of about 1 to about 3.

According to another exemplary implementation, the labeling of the analyte-binding partners with the labeling substance to prepare a labeled conjugate solution may comprise the steps of: determining a minimum stabilizing concentration of the analyte-binding partners; and preparing a conjugate solution containing the metal nanoparticles (NPs) (for example: gold NPs) encapsulated nanocomposite complex and minimum concentration of analyte-binding partners. In another exemplary embodiment, the analyte-binding partners may be immobilized on the surface of the gold nanoparticles.

In another exemplary implementation, determining a minimum stabilizing concentration of the analyte-binding partners, may take place via: mixing an electrolyte with the synthesized gold nanoparticle encapsulated nanocomposite complex and various concentration of the analyte-binding partners. According to one instance, the minimum stabilizing concentration of the analyte-binding partners may be in the range of about 5 μg to about 8 μg in one milliliter of the labeling substance.

According to another exemplary implementation of the present disclosure, preparing a conjugate solution containing the metal NPs encapsulated nanocomposite complex may be implemented through steps of: adjusting the PH of the labeling substance solution to obtain a specific PH which may be in the range of 7-9; mixing a solution containing minimum concentration of the analyte-binding partners, the labeling substance solution, and at least one stabilizing agent; Incubating the mixture; removing non-immobilized analyte-binding partners; and adjusting PH and then storing the solution. Disclosed conjugation solution, is applied into a pad by for example, saturating the pad in the solution and finally drying the pad.

In another exemplary implementation, the conjugation pad prepared according to the exemplary steps mentioned above, may join through, for example adhering, to a control pad which contain test zone and control zone, having different immobilized analyte-binding partners. The test strips which includes a detection pad that was prepared according to exemplary embodiments consistent with the present disclosure, further contain a sample pad to capture the sample and transfer it to the conjugation pad and a sample plate.

In another exemplary implementation, in addition of determining the presence of an analyte qualitatively by observing the creation of the test line, the amount of the analyte may also be determined semi-quantitatively. Semi-quantitatively approach the amount of the analyte is determined based on the broadness o the created test line.

In another exemplary implementation, the present pad may determine the analyte when they the concentration of the analyte is more than 100 μg/ml.

### BRIEF DESCRIPTION OF THE DRAWINGS

FIG. 1 illustrates method for synthesizing a conjugated solution, consistent with one or more exemplary embodiments of the present disclosure.

FIG. 2 (related art) illustrates operation of lateral flow immune-chromatographic assay for detecting the presence of an analyte, consistent with one or more exemplary embodiments of the present disclosure.

FIG. 3A illustrates infra-red (IR) spectra of an opened multi-walled carbon nanotube (MWCNT), consistent with one or more exemplary embodiments of the present disclosure.

FIG. 3B illustrates infra-red (IR) spectra of MWCNT-g-PCA, consistent with one or more exemplary embodiments of the present disclosure.

FIG. 3C illustrates infra-red (IR) spectra of MWCNT-g-PCA-Au, consistent with one or more exemplary embodiments of the present disclosure.

FIG. 4 illustrates the absorption spectra change during progression of the complexation between Au and MWCNT-g-PCA, consistent with one or more exemplary embodiments of the present disclosure.

FIG. 5A illustrates the transmission electron microscope (TEM) images of CNT-g-PCA-Au with an image resolution of 75 nanometers, consistent with one or more exemplary embodiments of the present disclosure.

FIG. 5B illustrates the transmission electron microscope (TEM) images of CNT-g-PCA-Au with an image resolution of 50 nanometers, consistent with one or more exemplary embodiments of the present disclosure.

FIG. 5C, illustrates the transmission electron microscope (TEM) images of CNT-g-PCA-Au with an image resolution of 60 nanometers, consistent with one or more exemplary embodiments of the present disclosure.

FIG. 6 illustrates the change in optical properties of stabilized gold sol at 580 nm after addition of various amounts of polyclonal anti-TMV antibodies and NaCl solution to colloidal gold solution, consistent with one or more exemplary embodiments of the present disclosure.

FIG. 7 schematically illustrates an example implementation of a procedure of binding prepared labeled detection reagent with the analyte, consistent with one or more exemplary embodiments of the present disclosure.

FIG. 8 illustrate Lateral-flow immune-dipsticks using anti-TMV—MWCNTs g-PCA-Au, [Control line (goat anti-rabbit IgG); the bottom line: test line (anti-TMV)]: kit (a); test in PBS (b); virus concentration: 150 ng/ml (c); 250 ng/ml (d); and 500 μg/ml (e), consistent with one or more exemplary embodiments of the present disclosure.

FIG. 9 illustrates Lateral-flow immune-dipsticks using anti-TMV-gold nanoparticles: virus concentrations: 250 ng/ml (a); and 500 μg/ml (b), consistent with one or more exemplary embodiments of the present disclosure.

### DETAILED DESCRIPTION

In test strip assays a ligand bound by a visually detectable solid support may be measured qualitatively (and in some cases semi-quantitatively). Therefore, while test strips may employ any one of a variety of assay schemes, including sandwich assays (both direct and indirect) and competitive reaction assays, all have in common the element of a detectable label or labeled detection agent that permits identification of an analyte of interest when present in an experimental sample. The lateral flow immunoassay test strips are the most profitable in detecting disease and infection.

Exemplary embodiments consistent with the present disclosure include a method for preparing a detection pad for determining the presence of an analyte of interest. The detection pad may comprise of a conjugation pad and a control pad which are joined together. The conjugated pad contains detection reagent (or labeled detection reagent), in which, a labeling substance may be conjugated with analyte-binding partners. The labeling substance used in the conjugation pad may contain a plurality of metal nanoparticles which may be encapsulated into nanocomposites.

In an exemplary embodiment, the nanocomposites used in the present disclosure, are carbon nanotubes which may be grafted with poly (citric acids). After reacting the labeled detection reagent (applied in the detection pad), with a test sample which contains the analyte of interest, the analyte may then be detected by a variety of means. Since the nanocomposite containing metal particles are colored after reaction, visual detection of a positive result is possible. The detection pad prepared in the present disclosure, results in better detection and high sensitivity.

In another exemplary embodiment, the method for preparing the detection pad may include the steps of: preparing a labeling substance; labeling of the analyte-binding partners with the labeling substance to synthesize a detection reagent; preparing a control pad; applying the detection reagent in a conjugation pad; and joining the conjugate pad and the control pad to obtain the detection pad.

The terms “analyte” or “analyte of interest” are used herein interchangeably, and they may refer to: bacteria and viruses, including indicator bacteria for microbiological assays, infective disease agents; non-viable microorganisms; proteins, including immunoglobulins, TSH, FSH, hCG, LH, ferritin, CEA, PSA, insulin, hemoglobin, growth hormone, and C-reactive protein; peptides; carbohydrates; polymers; and other types of molecular and cellular entities.

The terms “analyte-binding partner” or “analyte-binding molecule” are used herein to refer to compounds having spatial and/or polar features which permit to bind to the analyte. The analyte-binding partners may include: antigens and antibodies, or fragments of antibodies, both polyclonal and monoclonal, lectins and carbohydrates, hormones and hormone receptors, enzymes and enzyme substrates, vitamins and vitamin binding proteins, drugs and receptors.

The terms “detective reagent”, “conjugated solution”, “conjugated complex”, “labeled detection reagent”, “labeled reagent compound”, or “analyte-labeled reagent compound”, which are used herein interchangeably, refer to a compound or complex in which, the analyte-binding partner is bounded and/or conjugated with the labeling substance and the resultant complex is used in the detection pad to detect the presence of an analyte of interest. Generally, reaction of an analyte with a detective reagent containing analyte-binding partners, which used on a reagent absorption pad, may generate a detectable signal.

The term “labeling substance”, used herein refers to a compound containing metal particles, such as gold nanoparticles, which after binding with analyte-binding partners' used in test zone, results the visual detection and therefor, positive results.

The term of “detection pad”, used herein refers to a pad in which the conjugated pad and the control pad have been joined to each other through, for example, adhering those pads.

The term “conjugation pad” used herein, refers to a pad, may be a capture membrane or porous pad, which contain labeled detection agent capable to bind with the transferred analytes from the sample pad. The bounded labeled detection reagent with the analyte of interest are transferred to the control pad having test and control zones. In some implementation, the conjugate pad may also include one or more stabilizing compounds that are able to induce thermal stability and also stability as to conditions imposed by humidity and temperature.

The term “control pad” used herein, may refer to a nitrocellulose strip pad having test zone and control zone. The term “test line” or “test zone” used herein, refers to a test zone in the sample pad which contain immobilized analyte-binding partner and the presence of the analyte of interest is detectable from test line through formation of a colorimetric signal. The sample containing the analyte of interest, results in producing a colorimetric signal in the test line. In fact, the nitrocellulose strip is able to absorb the sample from the conjugate pad and transfer the sample by capillary action to the test result zone and the control zone. The terms “control line” or “control zone” used herein, may be applied as a signal that implies the test functioned properly. The control zone may include a substance that binds to a different portion of conjugated solution than does the analyte.

The term ‘sample pad’ used herein, refers to a porous membrane which may be placed below, or anyhow, in contact with the conjugation pad. The aim of placing the sample pad is capturing the sample and transfer it to the conjugate pad by capillary flow.

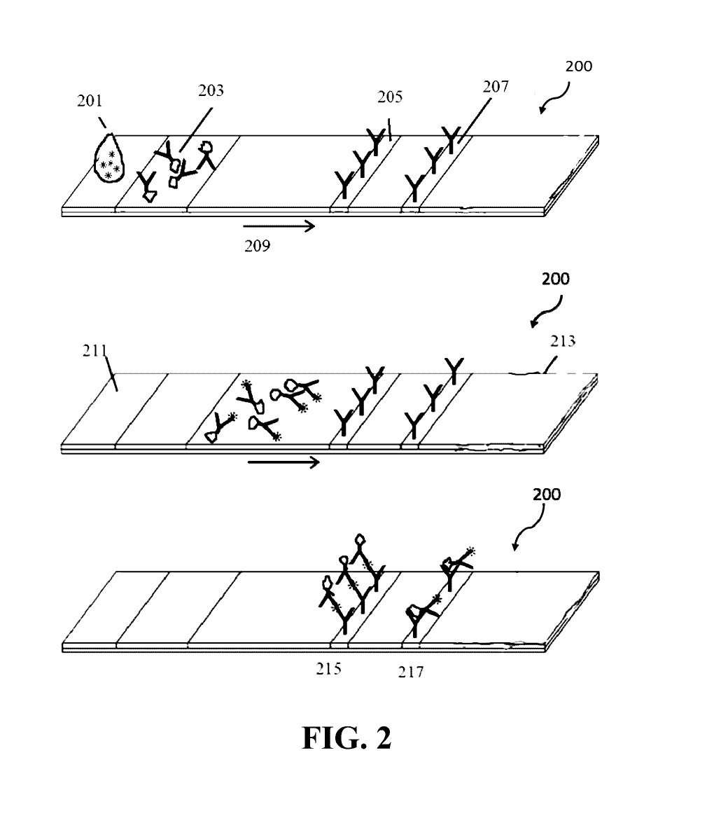

FIG. 2 (related art) illustrates a general view of an exemplary detection pad 200 and mechanism of its application. When liquid test sample 201 containing an analyte of interest, is applied to sample pad 211 of the detection pad, the sample travels through sample pad 211 and conjugate pad 203, by capillary action 209. When sample 201 travels through conjugate pad 203, sample 201 solubilizes the dried labeled molecule or detective reagent in conjugate pad 203. In the detection reagent, the labeling substance, has been conjugated with specific analyte-binding partners. If the analyte of interest is present in sample 201, the solubilized labeled molecule or detection reagent, binds the analyte of interest forming an analyte-labeled reagent complex. Otherwise, if the analyte of interest is not present in the sample, no complex is formed.

The analyte-labeled reagent complex in the case of a positive test, or the labeled reagent alone in the case of a negative test, then travels to control pad (nitrocellulose strip) 213, and travels through and passes test zone 205 and control zone 207 of the detection pad. Test zone 205 of the immunoassay device includes one or more immobilized molecules or reagents, such as antibodies, capable of specifically binding to the one or more analytes of interest or any portion of the analyte-labeled reagent complex.

If the analyte of interest is present in sample 201, the analyte-labeled reagent complex binds to the immobilized reagent of test zone 205, and forms a detectable line 215 there. If the analyte of interest is not present in the sample, no analyte labeled reagent complex is formed and therefore no binding occurs at test zone 205. Whether or not the analyte of interest is present in the sample to form the complex, the labeled detection reagent binds to the immobilized reagent of control zone 207, which doesn't contain the specific analyte-binding partner same as which in the test line, therefore no color change occurs or is observed. In fact, control zone 217 retains its color properties which do not change after reaction with analyte or the analyte-labeled reagent complex.

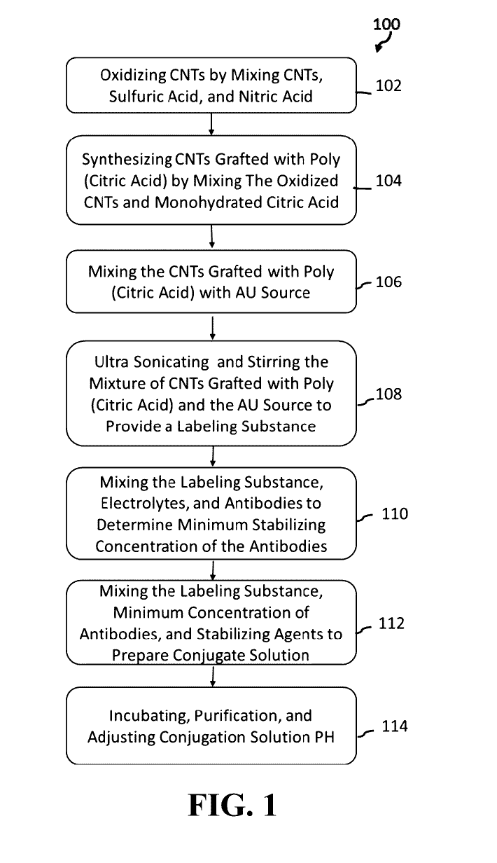

Referring now to FIG. 1, an exemplary method 100 for preparing a detection pad for use in a test strip to determine the presence of an analyte is illustrated, consistent with one or more exemplary embodiments of the present disclosure. Exemplary nanoparticles may be used by themselves or they may be used in combination with other metal nanoparticles. Preparing the pad containing detective reagent in which a labeling substance is conjugated with an analyte-binding partner to form a labeled detection reagent, may include the steps of oxidizing carbon nanotubes 102, in which sulfuric and nitric acid are mixed with the carbon nanotubes. The carbon nanotubes used in this application can be, for example, multi-walled carbon nanotubes (MWCNTs) or single-walled carbon nanotubes. The MWCNTs may be opened and functionalized using a sulfuric and nitric acid mixture (in the weight ratio of about 3/1). In this step, MWCNTs may be activated in nitric acid solutions to give carboxyl group modified MWCNTs (MWCNT-COOH).

According to step 104, citric acid polymerized onto functionalized carbon nanotubes by reaction of oxidized MWCNTs with monohydrate citric acid, resulting in hyper-branched poly (citric acid) grafted onto MWCNT (CNT-g-PCA). Both citric acid and functionalized MWCNTs contain alcoholic and acidic hydroxyl functional groups, hence citric acid may be polymerized onto functionalized MWCNT through poly-condensation reaction. In the next step 106, the prepared nanocomposite (MWNT-g-PCA) is mixed with a gold (AU) source. After certain steps, which may be for example, ultra-sonicating and stirring according to step 108, the AU nanoparticles (AUNPs) may be dispersed into the CNT nanocomposite and as a result, AUNPs may be encapsulated into an exemplary nanocomposite. In this exemplary method, gold nanoparticles may be encapsulated into the nanocomposite. However, any other nanoparticles which are able to use in the labeling substance or provide, for example a colorimetric signal in further steps, may be used. The Au source may be, for example, Chloroauric acid. (HAuCl4). However, any other AU source which is compatible for the reaction, may be used.

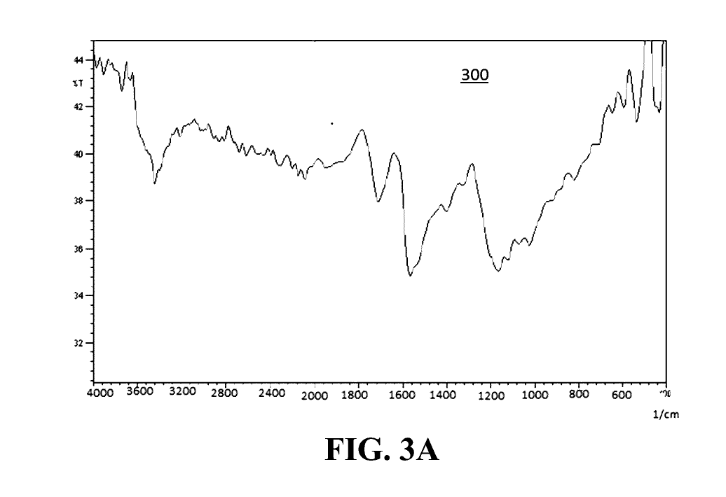

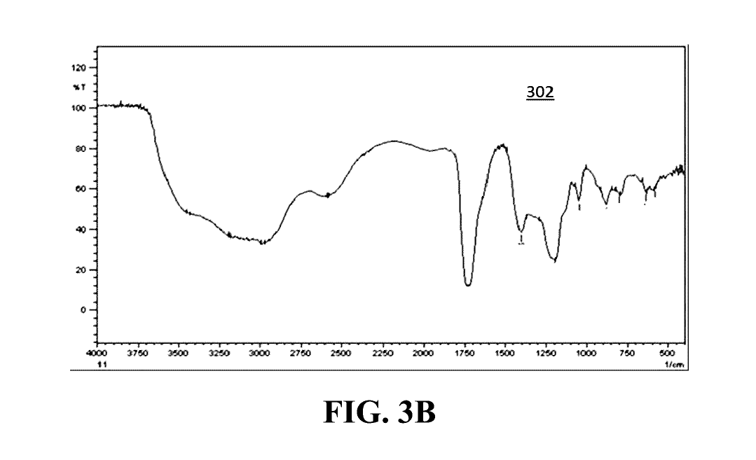

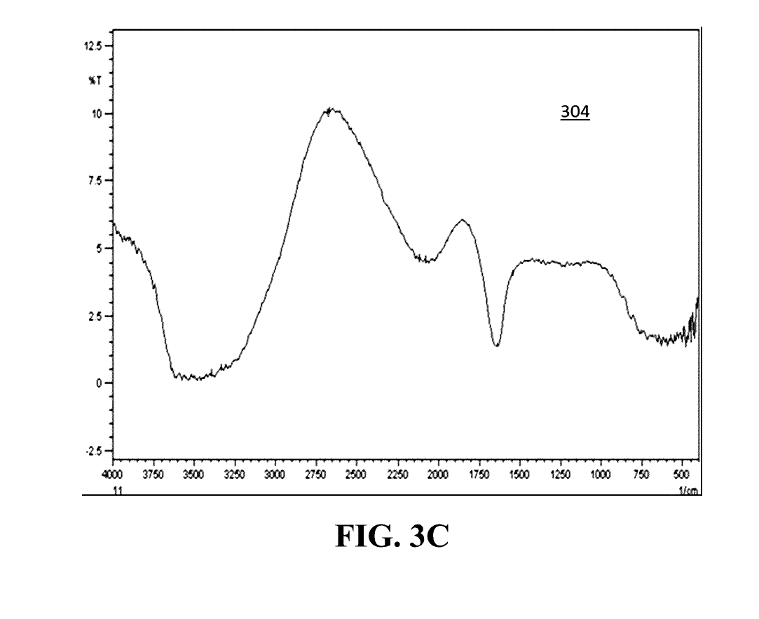

Referring now to FIG. 3A, the infra-red (IR) spectra of opened MWCNTs 300 is illustrated, consistent with one or more exemplary embodiments of the present dsiclosure. In this figure, the absorbance band of acidic and alcoholic hydroxyl functional groups of opened MWCNT appear at 3600-3200 cm⁻¹. The absorbance band at 1700 cm⁻¹ is related to the carbonyl groups of —COOH groups of opened MWCNT and two absorbance bands at 1590 and 1400 cm⁻¹ are related to the C═C aromatic bonds of MWCNT. As iFIG. 7 illustrated in FIG. 3B, which shows the IR spectra of MWCNT-g-PCA 302, the broad band at 2400-3530 cm⁻¹ is related to the acidic functional groups of grafted PCA onto the MWCNT. Because of overlapping of several absorbance bands, a relatively broad absorbance band may be observed at 1750-1700 cm⁻¹. These absorbance bands are related to the C═C bands and carbonyl groups of opened MWCNT and also acidic and esoteric carbonyl groups of grafted PCA. FIG. 3C shows the IR spectra of MWCNT-g-PCA-Au 304, in which immobilization of the gold nanoparticles on polymeric shell of CNT-g-PCA, with regard to the broadness of the bands, is confirmed.

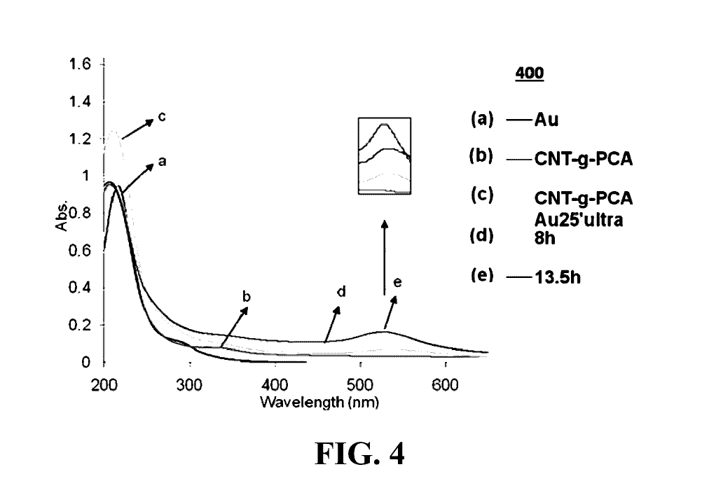

FIG. 4 shows the UV spectra 400 of complexation of Au and MWCNT-poly (citric acid) for different time period. The weak absorption peak centered at about 530 nm in the spectrum of MWCNT/gold NP composites was compatible with the reported values in the art for gold NPs. Enhancing the intensity of absorption bands during the time demonstrates gradual increases in the surface plasmon absorption by the colloidal gold particle in solution in which the concentration of the nanoparticle is increased due to the increasing of immobilized Au nanoparticles in the host nanocomposite.

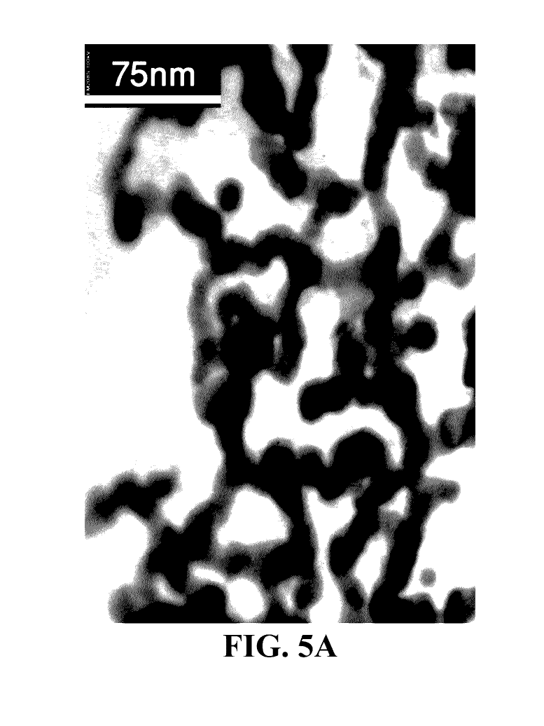

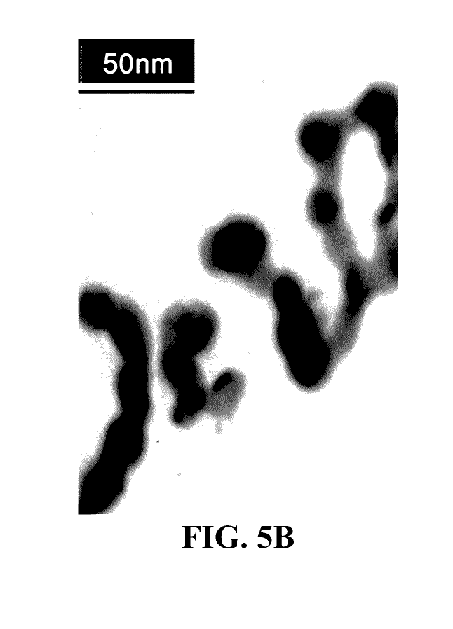

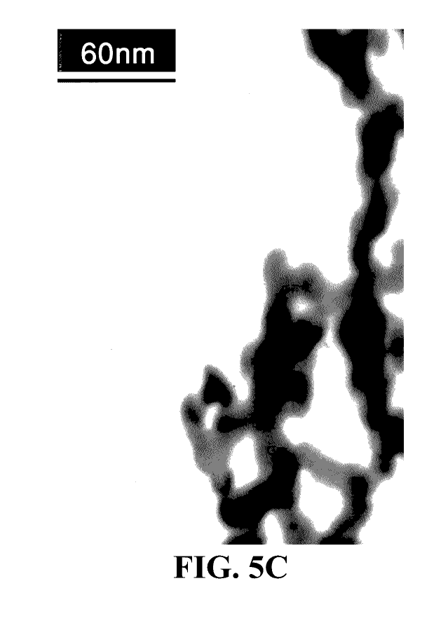

FIGS. 5A-C shows the transmission electron microscope (TEM) image of immobilized gold nanoparticles in the PCA shell of the CNT-g-PCA, consistent with one or more exemplary embodiments of the present disclosure. TEM images clearly show the presence of metal nanoparticles on the surface of MWCNTs. This method could not be limited to gold, and it may be used to assemble a variety of other metal NPs on the modified CNT surfaces. FIG. 5A illustrates the presence of gold NPs homogenously distributed in CNT-g-PCA-Au at resolution of 75 nanometers, while FIGS. 5B and 5C, illustrate the same at resolution of 50 nanometers and 60 nanometers, respectively. FIGS. 5A to 5C, approve this assumption that nanoparticles are placed on the surface of CNTs and are fitted on the CNT wall by PCA shell.

Referring back again to FIG. 1, the resulted AU encapsulated with MWNT-g-PCA according to step 108, may be considered a labeling substance, and therefore may be conjugated with a specific analyte-binding partner. In the next exemplary step, 110, the minimum concentration of analyte-binding partners may be determined. The minimal load of immobilized antibodies, may be determined on the basis of concentration of analyte-binding partners (e.g. antibodies) which bind to the gold nanoparticles encapsulated in the nanocomposite. Determining the minimum concentration of antibody may be used upon obtaining the antibody conjugates with gold nanoparticles.

In step 110, to determine the minimal concentration of analyte-binding partners, various amount of analyte-binding partners may be added to the Au nanoparticle encapsulated nanocomposite (MWCNTs-g-PCA-Au). In some exemplary embodiments, an electrolyte, for example NaCl solution, may be used in this step. Minimal stabilizing concentration of antibodies, may be estimated by the change in optical properties of stabilized gold sol at, for example 580 nanometers.

In the next exemplary step (112), a conjugate solution for forming reactive detection agent may be prepared by mixing the nanocomposite and the minimum concentration of Analyte-binding partners. In fact, during this step and further step (114), the analyte-binding partners may be labeled and immobilized into a solution to be used in a pad which is specifically designed to detect the presence of the analyte.

In some implementation, the prepared nanocomposites (MWCNTs g-PCA nanocomposites), may act as a substrate for the assembly of gold nanoparticles. MWCNT-g-PCA may be used as an affinity support for the immobilization of negatively charged gold nanoparticles. In exemplary embodiment, increasing the number of gold nanoparticles in the nanocomposite enhances the assay sensitivity of the developed immune-dipstick.

In some exemplary implementation, the PH is adjusted before mixing the analyte-binding partner with the gold NPs encapsulated nanocomposite, to be in the range of 7-9. In some additional exemplary implementation, the PH adjustment may take place after such mixing. In some exemplary implementation for immobilizing of the analyte-binding partners to the gold NP's surfaces, one or more stabilizer may be used. One exemplary substance that was used in the present application as stabilizing agent, is Bovine Serum Albumin (BSA). However, other immobilizing substance may also be used.

Next, the conjugate solution of the previous step (112), after various actions (according to step 114) which may include, for example, incubating, purification, and PH adjustment, become ready to be used as a labeled detection complex in a test pad.

The analyte of interest to be detected according to present application, may be any viral/bacterial proteins or allergens or other biological active agents. The “analyte-binding partner” which is conjugated with the labeling substance or the complex containing Au nanoparticles encapsulated into the MWNT-g-PCA nanocomposite, may be a biological active agent that is capable to bind and react with the analyte of interest in the sample.

In some implementation, the analyte-binding partner, may be selected from the group consisting of various biological active agent, including human or animal antibody or functional antibody fragment. The exemplary analyte which its presence in a sample are determined by the final prepared pad in the present disclosure, is selected from the viruses group. Since this application seeks to enhance the sensitivity of the pad, together with lowering cost compared to common methods, selection the kind of analyte and its specific binding partner is not essential step in the present application. This means, any kind of analyte-binding partner (antibody or functional antibody fragment) which may fulfill its intended function of specific binding to the analyte, can be applied in related preparation steps (110 and 112).

To prepare a pad containing the prepared labeled detection agent (conjugated pad), a capture membrane (porous pad), may be saturated in the conjugated solution prepared in step 114, and after drying, the dried pad may be adhered to a control pad (nitrocellulose strip) to place in a sample plate.

The term of “qualitatively” which used herein as one of the approaches that the prepared detection pad, may determine the presence of an analyte, is the way in which the presence of an analyte is determined through observing a test line in addition to the control line.

The term of “semi-quantitatively' used herein may be considered as another approach in which, determining of the presence of the analyte, in addition to determining the amount of the analyte (semi-quantitatively” may tack place. Determining the amount in this approach is based on the broadness and clearness of the test line and compare it with a prepared index in which, each broadness and clearness have been defined as a special amount or range.

The following examples represent methods and techniques for carrying out aspects of the present disclosure. It should be understood that numerous modifications could be made without departing from the intended scope of the disclosure.

### EXAMPLE 1

### Preparation of Labeling Substance

### Preparation of MWCNT-g-PCA Nanocomposites

CNTs were opened and citric acid polymerized onto functionalized carbon nanotubes. Briefly, MWCNTs were activated in nitric acid solutions to give carboxyl group modified MWCNTs (MWCNT-COOH), followed by reaction with monohydrate citric acid, resulting in hyper-branched poly (citric acid) grafted onto MWCNT (CNT-g-PCA).

Multiwall carbon nanotubes were opened and functionalized using a sulfuric and nitric acid mixture (in the ration of about 3/1) Both citric acid and functionalized MWCNTs are containing alcoholic and acidic hydroxyl functional groups, hence citric acid may be polymerized onto functionalized MWCNT through poly-condensation reaction. FIG. 3A shows the infra-red (IR) spectra of opened MWCNT. In this figure, the absorbance band of acidic and alcoholic hydroxyl functional groups of opened MWCNT are appeared at 3600-3200 cm⁻. The absorbance band at 1700 cm⁻¹ is related to the carbonyl groups of —COOH groups of opened MWCNT and two absorbance bands at 1590 and 1400 cm⁻¹ are related to the C═C aromatic bonds of MWCNT. In the IR spectra of MWCNT-g-PCA (FIG. 3B), the broad band at 2400-3530 cm⁻¹ is related to the acidic functional groups of grafted PCA onto the MWCNT. Because of overlapping of several absorbance bands, a relatively broad absorbance band may be observed at 1750-1700 cm⁻¹. These absorbance bands are related to the C═C bands and carbonyl groups of opened MWCNT and also acidic and esoteric carbonyl groups of grafted PCA.

### Preparation of MWCNT-g-PCA Nanocomposites Containing Au Nanoparticles

The prepared MWCNT-g-PCA composites were mixed with moderate concentration Chloroauric acid (HAuCl4) and placed in an ultrasonic bath for 20 min in order to well disperse metal ions in the polymeric shell of nanocomposite. The solution was then stirred at room temperature for 8 hours. The effect of reaction parameters (such as pH, temperature, ultrasonic time) on the synthesis of metal nanoparticles with MWCNT-g-PCA was studied by UV-vis experiments.

### EXAMPLE 2

### Labeling the Analyte-binding Partner with the Labeling Substance

Labeling Antibody with MWCNTs-g-PCA-Au

To obtain MWCNTs-g-PCA-Au conjugates with heterogeneous protein molecules of antibodies as analyte-binding partners, optimal synthesis conditions (such as pH, ionic strength of solution, and protein concentration (or antibody preparation)), were optimized in the sol. The minimal stabilizing concentration of antibodies was estimated by the change in optical properties of stabilized gold sol at 580 nm after addition to the MWCNTs-g-PCA-Au solution of various amounts of antibodies and NaCl solution. Non-stabilized MWCNTs-g-PCA-Au solution color is changed in the range of antibody concentration of 0-5 μg/ml due to coagulation of sol particles.

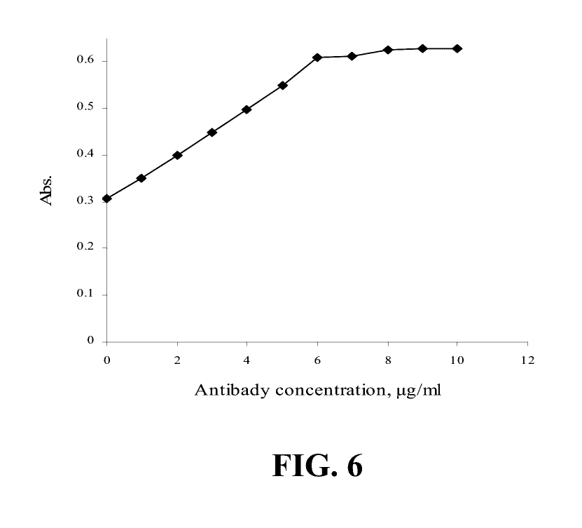

FIG. 6 illustrates that in the studied range of sol loading by antigens of 1-10 μg/ml sol 600, the minimal stabilizing concentration of immobilized antibodies is in the range of about 5 to about 8 μg/ml, which is revealed by the plateau at 580 nanometers of the MWCNTs-g-PCA-Au system optical density dependence on concentration of immobilized antibodies. In this case the gold sol which stabilized by antibodies color did not change after addition of electrolyte solution. Finally, antibody concentration for immobilization on the gold nanoparticles was chosen according to the dependence of the MWCNTs-g-PCA-Au solution optical density at 580 nm on antibody concentration, based on the criterion of 10% excess over the level providing for the plateau of optical density. To fulfill this requirement, the antibody concentration of about 7 μg/ml MWCNTs-g-PCA-Au solution was chosen.

### EXAMPLE 3

### Preparing Conjugate Solution

In general, the analytical performance of the membrane-based dipstick immunoassay may be affected by using different parameters, e.g. the type and pore size of the membrane, type of absorbent pad, and concentration of blocking reagent. The exemplary analyte used in the present disclosure is Tobacco Mosaic Virus (TMV), and the analyte-binding partner is anti-TMW. To obtain high sensitivity the amount of Anti-TMV—PBS (0.01 Molar, at pH of about 7.5) immobilized at the test line was chosen to be about 2.0 μl/0.5 cm of Anti-TMV—(0.25 mg/ml).

For preparing the exemplary conjugate solution (detective reagent), 1 ml MWCNTs-g-PCA-Au solution with A520=1.0 was titrated with 0.1 molar K2CO3 to pH 8.5, and 14 μl antibody solution (0.5 mg/ml) was added. Minimal stabilizing concentration of polyclonal antibodies of about 7 μg/ml was chosen. Bovine serum albumin (BSA) was added to final concentration 0.1% for additional stabilization of the resulting polyclonal antibody complex with colloidal gold nanoparticles. The mixture was stirred for 2 hours at a cold water bath to allow antibodies adhesion to the gold nanoparticle surface. After 2-hour incubation at 4° C. to remove non-bound antibodies, the mixture was centrifuged for 45 min at 10,000 rpm and 4° C. The pellet was re-suspended in 0.05 M K-phosphate buffer, pH 7.5 (containing 5% sucrose, 1% BSA and 0.5% Tween 20). The resulting antibody—MWCNTs-g-PCA-Au conjugate (IgG-MWCNTs g-PCA-Au) was stored at 4° C.

The detection principle of the one-step lateral-flow immune-dipstick, is based on the competitive immunoreaction between Anti-TMV (test line) and the TMV (as the analyte of interest in the present application) in the sample solution for the limited antibodies on the MWCNTs g-PCA-Au surface. The TMV—anti-TMV—MWCNT-g-PCA-Au were formed in a micro-well by a simple pre-incubation of bio-nanocomposites and TMV analyte in the sample solution. The positive or negative results as obtained by the dipsticks relied on visible pink color of the test and control lines. If the TMV concentration of the sample was higher than the cutoff value (sample considered positive), both lines were colored. In contrast, only a colored control line appeared if the TMV concentration in the sample is lower than the cutoff value (sample considered negative). Concluding, the control line should be always visible regardless of the concentration of the analyte.

### EXAMPLE 4

### Preparation of Immunochromatographic Test Strips

Nitro-cellulose Membrane was cut into 2.5 cm×0.5 cm sections. Test line (on the bottom of the membrane) was coated with 1 μl Anti-TMV diluted in 1 μl 0.01 M PBS buffer (pH 7.5) with sampler. The control line (on the top of the membrane) was coated with 1 μ1 of goat anti-rabbit IgG diluted in 1 μl 0.01 M PBS buffer (pH 7.5). The distance between the test line and control line was 0.5 cm. The test strips were dried at room temperature for 1 hour and blocked with 2% (w/w) casein/PBS (0.05 M, pH 7.5) for another 30 min at room temperature. It was then washed with PBS and again dried for 2 hours at room temperature.

The conjugate pad (0.5×1 cm) was saturated with 30 μ1 IgG-MWCNTs g-PCA-Au and then dried at room temperature for 2 hours. Finally, filter paper as a sample pad and a conjugation pad (0.5×1 cm) were adhered onto the plate to complete the fabrication of the device.

### EXAMPLE 5

### Assay Procedure

The extracted dilute virus sample was placed in a test tube. The dipstick was dipped in the virus solution at the sample pad side. Driven by capillary forces, the liquid migrated along the dipstick into the absorbent pad. The IgG-MWCNTs g-PCA-Au (detector reagent), which was solubilized from the conjugate pad by re-dissolving in the extract solution, reacted with TMV (if it was present in the extract), while the whole complex was migrating along the membrane.

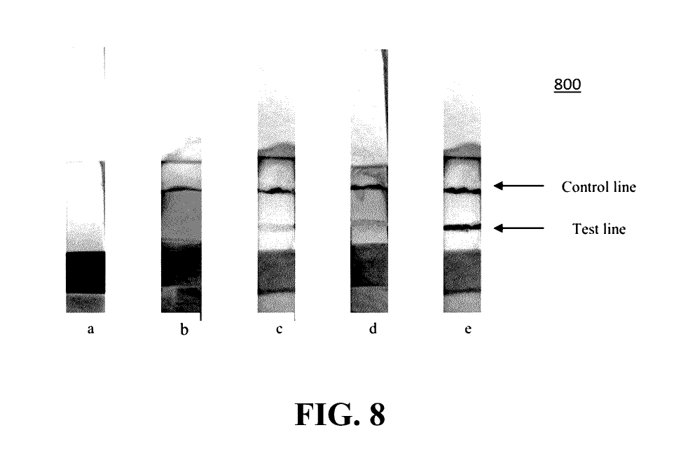

Referring now to FIG. 8, a Lateral-flow immune-dipsticks using anti-TMV—MWCNTs g-PCA-Au 800 is illustrated. In this figure, the upper line is the control line (containing goat anti-rabbit IgG); and down line is the test line (containing anti-TMV). In this figure, (a) is the prepared kit before contacting to any sample, the kit denoted as (b), is the kit tested in PBS. These two kit: [(a) and (b)] are used in this figure to illustrate the change in test line using various concentration of the viruses. The various virus concentration used in this example to show the sensitivity of the prepared sample pad. The concentration of 150 μg/ml virus in sample is illustrated in the kit denoted as (c), the concentration of 250 μg/ml virus in the sample indicates as (d) and the concentration and 500 μg/ml virus in the sample symbolized by (e). The color intensity of the test line inversely correlated with TMV concentration in the sample. After 5 min, the test result was visually evaluated. The positive test resulted in two red lines (test and control lines). The positive test means the analyte (TMV) in the sample is detectable in this concentration. The negative samples (no virus detect in this concentration by the pad) gave only one red line (control line). If no control line was present, the test was considered to be invalid.

In the present application, the cutoff value was defined as the lowest TMV concentration, which inhibited apparent color development at the test line. FIG. 8 illustrated that virus through the concentration of 150 ng/ml was detected by the conjugate CNT-g-PCA/Au-IgG. As illustrated in FIG. 8, by increasing the concentration of the viruses, the color of the test line gets bold. This finding confirms that the prepared test not only may be used to determine the presence of an analyte, but also may be used for determining the amount of them in the sample as semi-quantitatively.

For verifying the enhanced sensitivity obtained by the prepared pad in the present application compared to conventional pads which normally contain pure gold nanoparticles or gold sol, two sample pads were prepared and the results may be compared by the results illustrated in the FIG. 8.

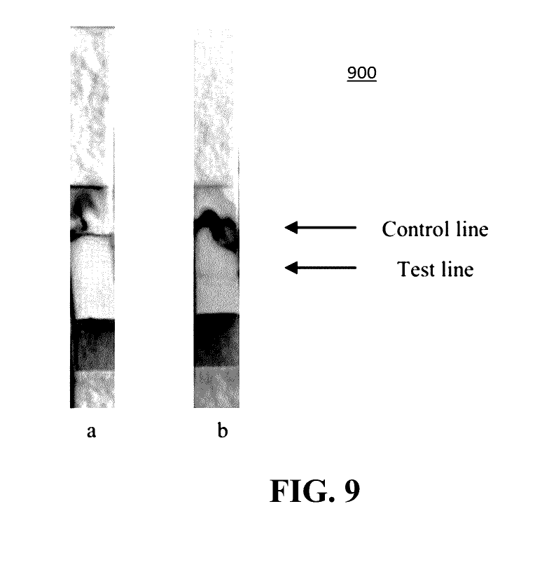

FIG. 9 illustrates Lateral-flow immune-dipsticks 900 using anti-TMV-gold nanoparticles: virus concentrations: 250 ng/ml (a); and 500 μg/ml (b), consistent with one or more exemplary embodiments of the present disclosure. When the pad containing pure gold nanoparticles (Au sole) contact with the samples containing 250 μg/ml virus (the pad which is denoted by (a), no line is observed in the test line. The test line only observed in the case of using the pad containing Au sol whence the sample contains 500 μg/ml virus concentration (the sample pad which denoted by (b). In fact, the minimum concentration of 500 μg/ml was detected and lower concentration was not detectable. This finding reveals that using Au conjugate in pad for virus detection, needs higher virus concentration in the sample and therefore, has the lower sensitivity than conjugated pad containing Au encapsulated in the nanocomposite, which its preparation was disclosed in the present application. Comparison of FIG. 8 and FIG. 9, illustrates that the test line of 500 μg/ml in FIG. 9 Is hardly detectable compared to the test line of 150 ng/ml in FIG. 8.

As a result, the cutoff value of conventional strip with pure gold nanoparticles as detection reagent is at least 500 μg/ml TMV, while the cutoff value with MWCNT-g-PCA-Au as a detection reagent is 3500 times lower, in the range of about 100 to about 150 ng/ml TMV. This results, approve higher sensitivity of the prepared detection pad disclose in the present application. The mechanism of color change of dipstick, when it is placed in a plant extracted solution as an example of biological solution, the virus particles move using the capillary effect so that the antibody marked viruses interact with Au nanoparticles and this migration continues by reaching to antibody test line, the virus builds up a complex with antibody and because of aggregation of au nanoparticles the test line gets colored. When other particles migrate and reach the control line, the existing antibody from the other way builds up a complex with labeled antibody so that the control line gets colored. FIG. 6 shows the mechanism of dipstick with the conjugate of the CNT-g-PCA/Au-IgG.

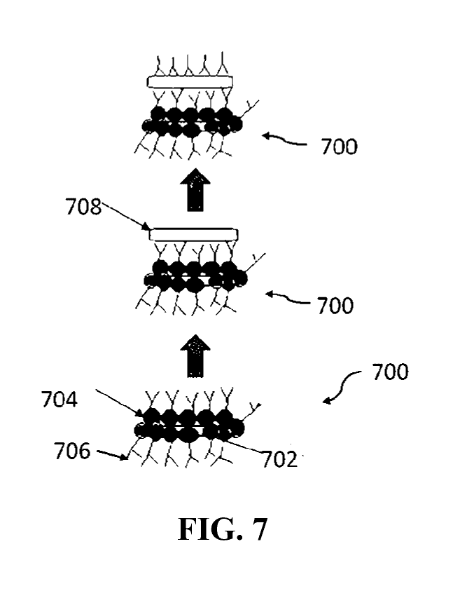

FIG. 7 schematically illustrates an example implementation of a procedure in which prepared labeled detection reagent 700 bonded with the analyte 708. Referring to this figure, encapsulation of gold nanoparticles 704 and good coverage of them by a nanocomposite 702, result in preparation of the labeling substance according to present disclosure MWCNTs g-PCA. After binding to an analyte binding partner 706 (which can be for example an antibody, the labeled detection reagent 700 according to an example implementation of the present disclosure can be prepared. After introducing an analyte 708, for example viruses, to the labeled detection reagent 700, CNT-g-PCA/Au-IgG according to an implementation of the present disclosure, strong binding occurred that may lead to the analyte 708 better identification.

With referring now to FIG. 5, we understand that the whole surface of the exemplary nanotube is covered monotonously by Au nanoparticles which may be connected to the antibodies. According to the virus length which also is about 200 micrometers, multiple and strong connections between viruses and antibodies placed on the surface of the nanotubes are supposed. This confirms the result observed in FIG. 8, which means bolder color and better detection by the prepared pad in the present disclosure during the test.

As a result, the increased sensitivity is likely attributable to the large number of gold nanoparticles contained in each MWCNTs g-PCA-Au. Regardless binding of only a few antibody molecules (anti-TMV) on the MWCNTs g-PCA-Au surface with the corresponding antigen (analyte), all of the carried nano-gold particles could participate in the dipstick response, and enhance the sensitivity of the membrane-based lateral-flow immune-dipstick.

What is claimed is:

1- A method of preparing a detection pad, comprising the steps of:
preparing a labeling substance, wherein the labeling substance comprises a plurality of metal nanoparticles encapsulated in a nanocomposite complex;
conjugating the labeling substance with at least one analyte-binding partner to prepare a labeled detection reagent;
applying the prepared detection reagent into a pad to prepare a reagent absorption pad;
preparing a control pad, wherein the control pad comprises of a nitrocellulose strip pad comprising a test zone and a control zone; and
binding the control pad and conjugation pad to obtain the detection pad.

2- The method according to claim 1, wherein the step of preparing the labeling substance includes synthesizing a nanocomposite complex, wherein the plurality of metal nanoparticles is encapsulated in the nanocomposite.

3- The method according to claim 2, wherein synthesizing the nanocomposite complex comprises the steps of:
synthesizing a nanocomposite containing carbon nanotubes grafted with poly citric acid;
mixing a metal source with the synthesized nanocomposite containing carbon nanotube grafted with the polycitric acid to provide a mixture; and
ultra-sonicating and stirring the mixture.

4- The method according to claim 3, wherein synthesizing a nanocomposite containing carbon nanotubes grafted with polycitric acid comprises:
oxidizing carbon nanotubes; and
mixing the oxidized carbon nanotubes and monohydrated citric acid to synthesize carbon nanotubes grafted with the poly citric acid.

5- The method according to claim 4, wherein oxidizing the carbon nanotubes comprises mixing the carbon nanotubes with nitric acid and sulfuric acid.

6- The method according to claim 5, wherein the ratio of the nitric acid to the sulfuric acid is in the range of 1 to 3.

7- The method according to claim 1, wherein the plurality of metal nanoparticles is selected from the group consisting of gold or silver nanoparticles or combination thereof.

8- The method according to claim 2, wherein the metal nanoparticles are gold nano particles (Gold NPs).

9- A method of claim 3, wherein the metal source is an Au source.

10- The method according to claim 7, wherein the Au source comprises a chloroauric acid (HAuCl4).

11- The method according to claim 1, wherein labeling of the analyte-binding partners with the labeling substance to prepare a labeled conjugate solution includes the steps of:
determining a minimum stabilizing concentration of the analyte-binding partners; and
preparing a conjugate solution containing the gold encapsulated nanocomposite complex and minimum concentration of analyte-binding partners, wherein the analyte-binding partners are immobilized on the surface of the gold nanoparticles.

12- The method according to claim 10, wherein determining a minimum stabilizing concentration of the analyte-binding partners comprises a step of mixing an electrolyte, the synthesized gold nanoparticle encapsulated nanocomposite complex, and various concentration of the analyte-binding partners.

13- The method according to claim 11, wherein the minimum stabilizing concentration of the analyte- binding partner is in the range of 5 μg to 8 μg in one milliliter of the labeling substance.

14- The method according to claim 10, wherein preparing a conjugate solution containing the gold encapsulated nanocomposite complex comprises the steps of:
adjusting the PH of the labeling substance solution to reach a specific PH;
mixing a solution containing minimum concentration of the analyte-binding partners, the labeling substance solution, and at least one stabilizing agent;
incubating the mixture;
removing non-immobilized analyte-binding partners; and
adjusting pH of the prepared conjugated solution containing Au nanoparticles.

15- The method according to claim 14, wherein the specific PH is in the range of 8 to 9.

16- The method according to claim 1, wherein the test strip comprises a sample pad, a conjugate pad, a control pad, and a sample plate.

17- A method of claim 16, wherein the conjugation pad joins to the control pad from the bottom, wherein the conjugation pad is partly overlapped by the control pad.

18- The method according to claim 16, wherein the control pad is a capture membrane containing a control zone and a test zone, wherein the test zone having the immobilized analyte-binding partners.

19- The method according to claim 1, wherein applying the conjugation solution into the conjugated pad comprises the steps of:
saturating the pad into the conjugated solution; and
drying the pad.

20- The method according to claim 1, wherein the analyte-binding partners is selected from the group consisting of: antigens and antibodies, fragments of antibodies, lectins, carbohydrates, hormones, hormone receptors, enzymes, enzyme substrates, vitamins, vitamin binding proteins, drugs and receptors.

21- The method according to claim 1, wherein the present of the analyte is determined qualitatively and semi-quantitatively.

22- The method according to claim 1, wherein the present of an analyte is detected in a concentration of more than 100 μg/ml.

23- A conjugation pad for use in a test strip assay to determine the presence of an analyte, wherein the conjugation pad comprises a labeling substance conjugated with an analyte-binding partner, wherein the labeling substance containing a nanocomposite and a plurality of metal nano particles.

24- The pad according to claim 23, wherein the plurality of nano particles are encapsulated into the nanocomposite.

25- The pad according to claim 23, wherein the nanocomposite is carbon nanotubes grafted with poly citric acid.

26- The pad according to claim 23, wherein the metal nanoparticles are selected from the group consisting of gold nanoparticles, silver nanoparticles and/or combination thereof.

27- The pad according to claim 23, wherein the nanoparticles are gold nanoparticles.

28- The pad according of claim 23, wherein the analyte-binding partners are immobilized on the surface of the gold nanoparticles.

29- The pad according to claim 23, wherein the analyte-binding partners is an analyte-binding partner capable of specific binding to the analyte.

30- The pad according to claim 23, wherein the analyte-binding partners is selected from the group consisting of: antigens and antibodies, fragments of antibodies, lectins, carbohydrates, hormones, hormone receptors, enzymes, enzyme substrates, vitamins, vitamin binding proteins, drugs and receptors.

31- The pad according to claim 21, further comprising one or more stabilizing compounds.
